(19) United States
(12) Patent Application Publication
(10) Pub. No.: US 2006/0020246 A1
(43) Pub. Date: Jan. 26, 2006

(54) Angiographic catheter for uterine artery embolization

(76) Inventor: Bruce McLucas, (US)

Correspondence Address: Robert A. Seldon, 18th Floor, 10940 Wilshire Blvd., Los Angeles, CA, 90024-3952, US

(21) Appl. No.: 10/897,877

(22) Filed: Jul. 22, 2004

(51) Int. Cl. A61M 25/00

(52) U.S. Cl. 604/264; 604/164.01

(57) ABSTRACT

An intra-arterial catheter for uterine artery embolization is disclosed comprising (a) an outer generally tubular catheter having a main body portion formed about a generally longitudinally-extending axis, and a distal generally tubular end region extending from said main body portion at an angle in the range of approximately 45° to and including approximately 75° and (b) an inner generally tubular catheter positioned for sliding movement within the outer catheter and terminating in a generally tubular cobra-shaped distal end region sufficiently flexible to fit for sliding movement within the outer catheter and to assume its cobra shape when extended beyond the outer catheter. Before use, the inner catheter is retracted within the outer catheter, and the catheters are inserted into the interior femoral artery. The inner catheter is deployed once the outer catheter reaches the aortic arch so that the cobra-shaped tip of the inner catheter can extend around the curve of the arch and enter the ipsilateral internal femoral artery where blood flow-blocking material can be discharged.

### FIELD OF THE INVENTION

This invention relates to catheters and, more particularly, to angiographic catheters for use in uterine artery embolization.

### BACKGROUND OF THE INVENTION

Fibroids are benign growths in the muscular wall of the uterus that can range in size from very small to quite large. Their known effects range from discomfort and backaches to interference with fertility.

There are many treatments available for women with fibroids. Medicine can shrink some fibroids, while surgery has been used in other cases. A relatively new way to treat women with fibroids is uterine fibroid embolization, which involves the cutting off of the fibroid's blood supply.

Fibroids require a supply of blood in order to grow, and will shrink or disappear completely when that supply is cut off. The embolization procedure entails the cutting of a tiny incision in the patient's groin region, and the passing of a small catheter through an artery to the uterus. In practice, a radio-opaque catheter is fed into the femoral artery, while x-ray imaging is used to locate the relevant to blood vessels and position the catheter. When the catheter is in place, a fluid containing tiny particles is injected into the artery via the catheter. The particles, typically made of plastic or gelatin sponge, are about the size of grains of sand, and are moved by the pressure from the heart into the smaller arteries that are supplying blood to the fibroid. The particles become lodged in those arteries, and block blood flow to the fibroid. Over time, the fibroids consequently shrink or disappear.

### SUMMARY OF THE INVENTION

An intra-arterial catheter for uterine artery embolization is disclosed comprising (a) an outer generally tubular catheter having a main body portion formed about a generally longitudinally-extending axis, and a distal generally tubular end region extending from said main body portion at an angle in the range of approximately 45° to and including approximately 75° and (b) an inner generally tubular catheter positioned for sliding movement within the outer catheter and terminating in a generally tubular cobra-shaped distal end region sufficiently flexible to fit for sliding movement within the outer catheter and to assume its cobra shape when extended beyond the outer catheter.

Before use, the inner catheter is retracted within the outer catheter, and the catheters are inserted into the interior femoral artery. The inner catheter is deployed once the outer catheter reaches the aortic arch so that the cobra-shaped tip of the inner catheter can extend around the curve of the arch and enter the ipsilateral internal femoral artery where blood flow-blocking material can be discharged. Further details concerning the invention will be appreciated from the following detailed description of the invention, of which the drawing is a part.

### THE DRAWING

In the drawing,

FIGS. 1A and 1B are schematic illustrations of a catheter constructed in accordance with the invention and shown in retracted and deployed configurations, respectively;

FIG. 2A is a schematic illustration showing the catheter of FIG. 1 as it approaches the aortic arch during use;

FIG. 2B is a schematic illustration showing the catheter of FIG. 1 with its inner catheter deployed to pass around the aortic arch and into the left common iliac artery during use in accordance with the invention;

FIG. 2C is a schematic illustration showing the catheter of FIG. 1 as it approaches the left uterine artery with its inner catheter retracted in accordance with the invention; and

FIG. 2D is a schematic illustration showing the catheter of FIG. 1 with its inner catheter deployed and entering the left uterine artery in accordance with the invention.

### DETAIED DESCRIPTION OF THE INVENTION

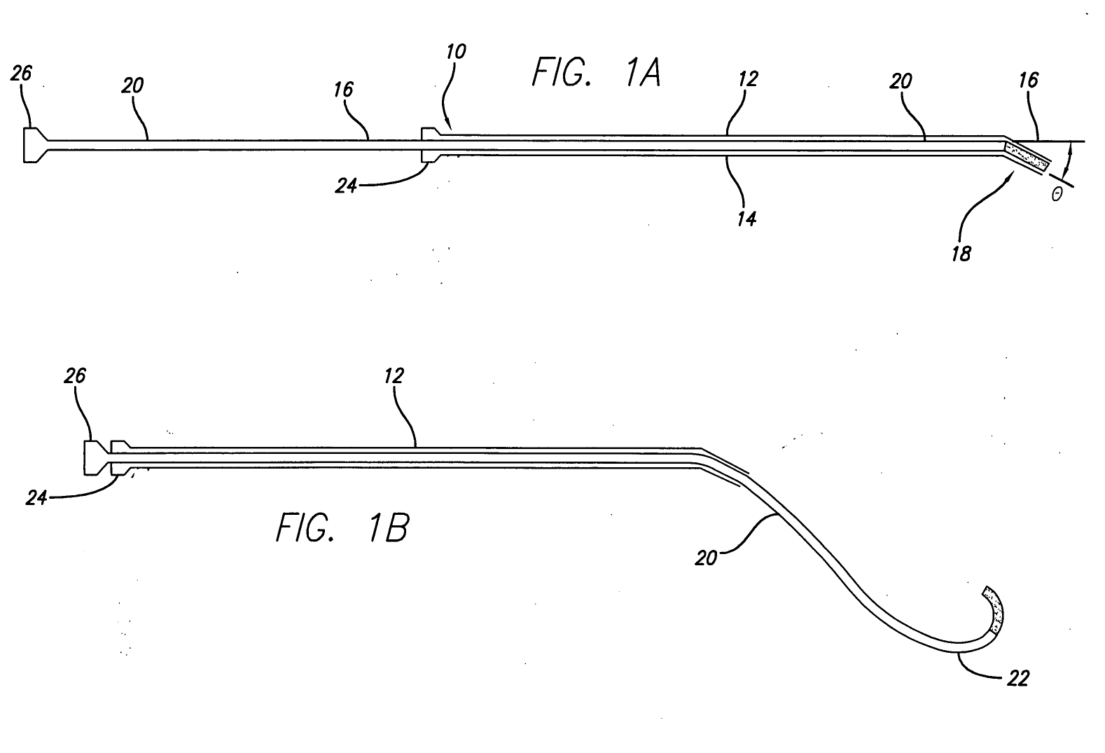

FIGS. 1A-1B are schematic illustrations of a catheter 10 constructed according to the invention. A catheter assembly 10 comprises an outer generally tubular catheter 12 having an elongated main body portion 14 approximately 50 cm in length with an outer diameter of approximately 5.5 mm formed about a generally longitudinally-extending axis 16. A generally tubular segment 18 extends from the distal end of said main body portion at an angle θ in the range of approximately 45°-75°, preferably about 60°, giving the outer catheter a hockey-stick appearance.

A generally tubular inner catheter 20 is positioned for sliding movement within the outer catheter 12 and terminates in a generally tubular cobra, or bowed, distal segment 22. Catheters with cobra tips are known, and further detail concerning the shape is accordingly omitted for the sake of brevity. The cobra-shaped segment 22 is sufficiently flexible to straighten sufficiently as the inner catheter is pulled back within the angled tubular segment 18 of the outer catheter to enable the inner catheter to lie completely within the outer catheter. The cobra body segment, however, possesses shape-memory that enables it to reassume its cobra shape when deployed. The outer diameter of the inner catheter 20 is preferably 4 mm, and the cobra-shaped tip is approximately 5 cm in length. Those skilled in the art will recognize that variations in the traditional “cobra” shape are possible without exceeding the scope of this invention; so long as the shape is generally the same, it is deemed to be a “cobra” shape within the meaning ascribed to that term herein.

The proximal ends of the outer and inner catheters terminate in respective handles 24, 26 that can be gripped by the treating physician and used to slide the inner and outer catheters relative to each other. A locking mechanism can be employed to retain the inner catheter within the outer catheter until the surgeon wishes to deploy the inner catheter. For example, the inner and outer catheters can be provided with inter-engaging surface features that retain the inner catheter at a particular position within the outer catheter until the surgeon wishes to deploy the inner catheter beyond the outer catheter. To permit such deployment, the blocking surface feature of the outer catheter can be overcome by the exertion of gentle distally-directed force on the inner catheter, while holding the outer catheter steady, to cause a movement or distortion of the surface features in a manner that permits distally directed movement of the inner catheter relative to the outer catheter.

The inter-engaging surface features of the inner and outer catheters can be any of any of a myriad of configurations. For example, they can comprise oppositely-extending bumps that protrude inwardly from the interior wall of the outer catheter and outwardly from the exterior surface of the inner catheter. The bumps on the inner and outer catheters can simply be formed in the catheter bodies'surfaces, and thereby possess the inherent flexibility required to get past each other upon application of the gentle distally-directed force. Naturally, other configurations employing inter-engaging bumps, ridges, grooves and combinations thereof, whether or not integrally formed in the catheters'surfaces, are possible and can be utilized are within the scope of the invention. This invention is not limited to the specific manner by which locking is accomplished, and it is intended that the scope of invention include all configurations and devices which provide the described function.

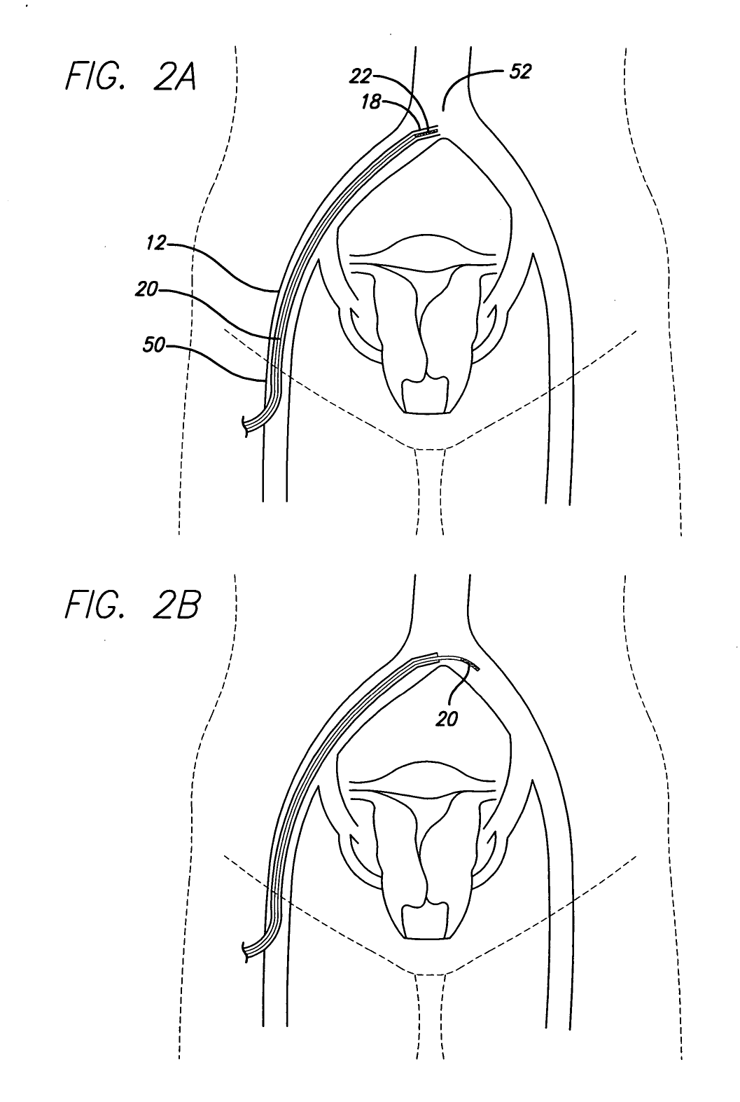

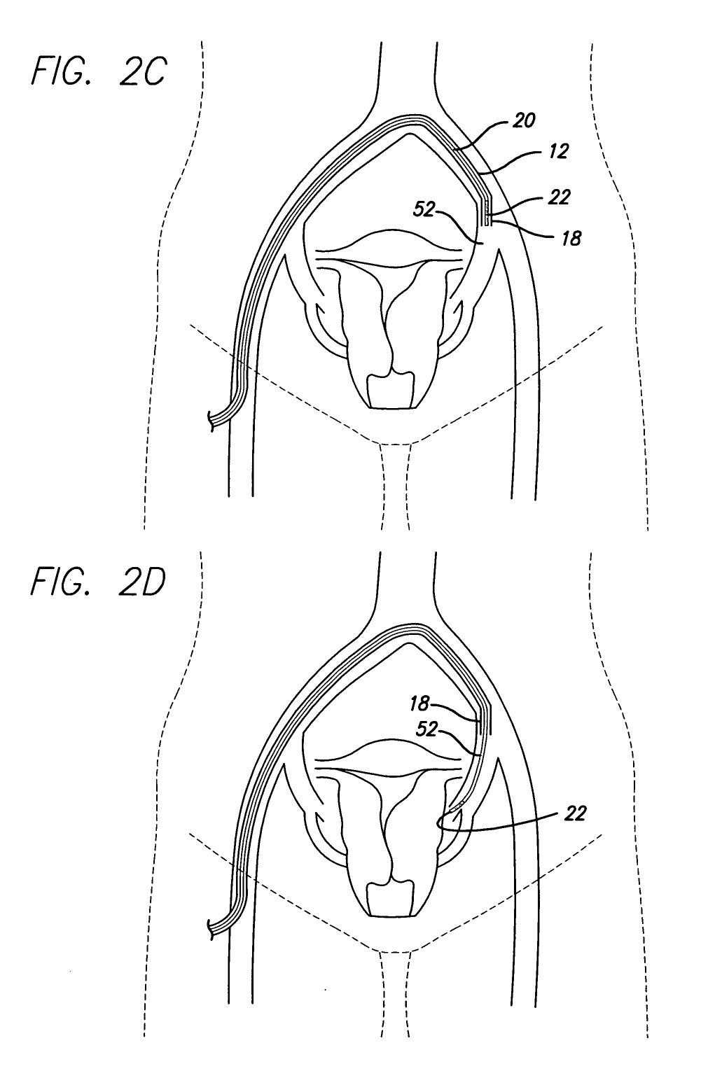

In use, the inner catheter is retracted within the outer catheter. As illustrated in FIGS. 2A-D, the outer catheter 12 is then fed through a small incision in the human patient into the femoral artery 50 and is guided towards the aortic arch 52, allowing the inner catheter to be readily moved through the artery as well. When the forward (i.e., distal) end of the outer catheter is positioned adjacent the angle to be traversed, as shown in FIG. 2B, the inner catheter 20 is advanced forwardly of the outer catheter by approximately 5 cm, permitting the inner catheter to resume its cobra shape and readily transverse the arch.

Once the inner catheter has been deployed, and its distal cobra-shaped segment 22 is properly positioned within the desired artery, blood-flow blocking material is dispensed into the desired artery in the usual manner from a syringe containing the blocking material in a suitable fluid, and coupled to the proximal end of the inner catheter by known means, such as a Luer lock. The material then travels through the catheter and into the artery, where the patient's blood flow carries the material to the site where blood flow is to be cut off.

Those skilled in the art will recognize that the particular manner by which the syringe is affixed to the catheter is not relevant to the scope of the invention, and that any means acceptable in the medical field is within the scope of the invention.

As illustrated in FIGS. 2C-2D, the described process is not restricted to use at the aortic arch, but can be employed at other locations as well. In FIGS. 2C-2D, the outer catheter is advanced to a position adjacent the left uterine artery 52 and the inner catheter is then deployed.

Although the present invention and its advantages have been described in detail, it should be understood that various changes, substitutions and alterations can be made herein without departing from the spirit and scope of the invention as defined by the appended claims.

I claim:

1. An intra-arterial catheter comprising:
an outer generally tubular catheter having a main body portion formed about a generally longitudinally-extending axis, and a distal generally tubular end region extending from said main body portion at an angle in the range of approximately 45° to and including approximately 75°;
an inner generally tubular catheter positioned for sliding movement within the outer catheter and terminating in a generally tubular cobra-shaped distal end region sufficiently flexible to fit for sliding movement within the outer catheter and to assume its cobra shape when extended beyond the outer catheter; and
locking means for retaining the distal end region of the inner catheter within the outer catheter until deployment is desired.

2. The catheter of claim 1 wherein the outer catheter has an outer diameter of approximately 5.5 mm.

3. The catheter of claim 1 wherein the inner catheter has an outer diameter of 4 mm.

4. The catheter of claim 1 wherein the distal end region extends from the main body portion at an angle in the range of approximately 55° to and including approximately 65o.

5. The catheter of claim 1 wherein the cobra shape is in the range of approximately 5 cm long to and including approximately 7 cm long it is direction of extension when deployed.

6. An intra-arterial catheter comprising:
an outer generally tubular catheter having a main body portion formed about a generally longitudinally-extending axis, and a distal generally tubular end region extending from said main body portion at an angle permitting it to demonstrate the origin of the uterine artery when inserted into the blood vessel of the
an inner generally tubular catheter positioned for sliding movement within the outer catheter and terminating in a generally tubular cobra-shaped distal end region sufficiently flexible to fit for sliding movement within the outer catheter and to assume its cobra shape when extended beyond the outer catheter; and
locking means for retaining the distal end region of the inner catheter within the outer catheter until deployment is desired.

7. An uterine fibroid embolization method comprising the steps of:
inserting into the artery of a patient a genereally tubular outer catheter having a main body portion formed about a generally longitudinally-extending axis, and a distal generally tubular end region extending from said main body portion at an angle in the range of approximately 45° to and including approximately 75°;
positioning the outer catheter adjacent the aortic arch;
transversing the aortic arch by sliding through the interior of the outer catheter a generally tubular inner catheter terminating in a generally tubular cobra-shaped distal end region sufficiently flexible to fit for sliding movement within the outer catheter and having a cobra shape when extended beyond the outer catheter; and
dispensing through the inner catheter a solution of particles sized to block the flow of blood in small arteries supplying blood to the fibroid.

8. The method of claim 7 wherein the inner catheter is advanced from the outer catheter by approximately 5 cm to transverse the aortic arch.

9. The method of claim 7 wherein the distal end region extends from the main body portion at an angle in the range of approximately 55° to and including approximately 65°.

10. The method of claim 1 wherein the cobra shape is in the range of approximately 5 cm to and including approximately 7 cm in length.

11. The method of claim 7 including the steps of withdrawing the inner catheter into the outer catheter after traversing the aortic arch and prior to dispensing the fluid, advancing the outer catheter to a position adjacent a subsequent artery, and deploying the inner catheter forwardly of the outer catheter into the said next artery.
